(19) United States
(12) Patent Application Publication
(10) Pub. No.: US 2022/0401028 A1
(43) Pub. Date: Dec. 22, 2022

(54) Nitinol Basket for Electrophysiological Mapping Catheter, and Systems, Devices, Components and Methods Associated Therewith

(71) Applicant: Ablacon Inc., Wheat Ridge, CO (US)

(72) Inventors: Peter Ruppersberg, Blonay (CH); Peter Jacobs, St. Louis Park, MN (US)

(21) Appl. No.: 17/685,192

(22) Filed: Mar. 2, 2022

Related U.S. Application Data
(63) Continuation-in-part of application No. 16/156,637, filed on Oct. 10, 2018, now abandoned.
Application No. 16/156,637 is a continuation-in-part of application No. 16/387,873, filed on Apr. 18, 2019, now Pat. No. 11,291,395.
Application No. 16/387,873 is a continuation-in-part of application No. 16/691,368, filed on Nov. 21, 2019.
Application No. 16/156,637 is a continuation-in-part of application No. 15/793,594, filed on Oct. 25, 2017, now Pat. No. 10,813,590.
Application No. 16/387,873 is a continuation-in-part of application No. 15/793,594, filed on Oct. 25, 2017, now Pat. No. 10,813,590.
Application No. 16/691,368 is a continuation-in-part of application No. 15/793,594, filed on Oct. 25, 2017, now Pat. No. 10,813,590.
Application No. 16/387,873 is a continuation-in-part of application No. 16/156,637, filed on Oct. 10, 2018, now abandoned.
(60) Provisional application No. 62/414,183, filed on Oct. 28, 2016.
(60) Provisional application No. 62/770,697, filed on Nov. 21, 2018.
(60) Provisional application No. 62/828,069, filed on Apr. 2, 2019.
(60) Provisional application No. 62/770,697, filed on Nov. 21, 2018.
(60) Provisional application No. 62/828,069, filed on Apr. 2, 2019.

(51) Int. Cl. A61B 5/00; A61B 5/287; A61B 5/367

(52) U.S. Cl. CPC A61B 5/6858; A61B 5/287; A61B 5/367; A61B 5/6859; A61B 2562/0209; A61B 2562/125

(57) ABSTRACT

Disclosed are various examples and embodiments of a Nitinol basket for an electrophysiological (EP) mapping catheter. In one embodiment, the Nitinol basket comprises a plurality of basket splines, each basket spline having a distalmost portion and a proximal end, where the distal tip is uninterruptedly contiguous and continuous with the distalmost portions of the basket splines and formed from the same piece, slab or ingot comprising Nitinol as the splines. In such an embodiment, the basket splines and distal tip are cut and formed from a same single length or piece of Nitinol tubing or a Nitinol hypotube. The respective distal portions of each of the Nitinol splines can be continuous and contiguous with, and connected to, the Nitinol distal tip, each spline being configured to extend outwardly away from an imaginary central axis of the Nitinol basket and its proximal end and distal portion to form a curved shape therebetween when the Nitinol basket is in an undeformed and deployed state. The splines can be configured to be spaced approximately equal distances apart from one another when the Nitinol basket is in an undeformed and deployed state, and the can be configured collectively to form a basket shape when the Nitinol basket is in an undeformed, expanded and deployed state.

### RELATED APPLICATIONS

This application claims priority and other benefits from each of the following provisional patent applications: (1) U.S. Provisional Patent Application Ser. No. 62/414,183 to Ruppersberg entitled “Electrophysiological Mapping Catheter” filed on Oct. 28, 2016 (“the '183 patent application”); (2) U.S. Provisional Patent Application Ser. No. 62/770,697 to Ruppersberg entitled “Electrophysiological Mapping Catheter” filed on Nov. 21, 2018 (“the '697 patent application”); and (3) U.S. Provisional Patent Application Ser. No. 62/828,069 to Ruppersberg entitled “Methods, Systems, Devices and Components for Electrophysiological Mapping Catheters” filed on Apr. 2, 2019 (“the '069 patent application”).

This application is also a continuation-in-part, and claims priority and other benefits from, each of the following pending utility patent applications: (4) U.S. Utility patent application Ser. No. 16/156,637 to Ruppersberg entitled “Multiple Configuration Electrophysiological Mapping Catheter, and Systems, Devices, Components and Methods Associated Therewith” filed on Oct. 10, 2018 (“the '637 patent application”); (5) U.S. Utility patent application Ser. No. 16/387,873 to Ruppersberg entitled “Systems, Devices, Components and Methods for Detecting the Locations of Sources of Cardiac Rhythm Disorders in a Patient's Heart and Classifying Same” filed on Apr. 18, 2019 (“the '873 patent application”); and (6) U.S. Utility patent application Ser. No. 16/691,368 to Ruppersberg entitled “Electrophysiological Mapping Catheter” filed on Nov. 21, 2019 (“the '368 patent application”).

Through the foregoing pending '637, '873 and '368 patent applications, this application further claims priority and other benefits from U.S. patent application Ser. No. 15/793,594 (now U.S. Pat. No. 10,813,590) to Ruppersberg entitled “Electrophysiological Mapping Catheter” filed on Oct. 25, 2017 (“the '594 patent application”). Each of the '637, '873 and '368 patent applications is a continuation-in-part of the '594 patent application. The '594 application claims priority from the foregoing '183 provisional patent application. The '873 and '368 patent applications each claim priority to both of the foregoing '697 and '069 provisional applications. The '873 patent application is a C-I-P of the '637 patent application.

Each of the foregoing '183, '697, '069, '637, '873, '368, and '594 patent applications is hereby incorporated by reference herein, each in its respective entirety.

### FIELD OF THE INVENTION

Various embodiments described and disclosed herein relate to the field of medicine generally, and more particularly to electrophysiological (EP) mapping systems, and EP mapping catheters, and techniques, procedures and methods associated therewith.

### BACKGROUND

Atrial fibrillation (or AF) is the most common type of heart arrhythmia or cardiac rhythm disorder. In atrial fibrillation, normal beating in the atria of the heart is irregular, and blood flow from the atria to the ventricles is compromised. Millions of people in the United States have AF. With the aging of the U.S. population, even more people will develop AF. Approximately 2% of people younger than age 65 have AF, while about 9% of people aged 65 years or older have AF. In some cases AF is treated with drugs. In other cases, external electrical shocks (electrical cardioversion) are delivered to the patient's heart. Open heart surgery can also be performed on a patient to treat AF.

Persistent atrial fibrillation (AF) is often caused by structural changes in atrial tissue, which can manifest themselves as multiwavelet re-entry and/or stable rotor mechanisms (see, e.g., De Groot M S et al., “Electropathological Substrate of Longstanding Persistent Atrial Fibrillation in Patients with Structural Heart Disease Epicardial Breakthrough,” Circulation, 2010, 3: 1674-1682). Radio frequency (RF) ablation targeting such host drivers of AF is generally accepted as one of the best therapeutic approaches to treating AF. RF ablation success rates in treating AF cases are currently limited, however, by a lack of sufficiently accurate and cost-effective diagnostic tools that are capable of quickly, cost-effectively, and precisely determining the source (or type), and location, of such AF drivers. Better diagnostic tools would help reduce the frequency and extent of cardiac ablation procedures to the minimum amount required to treat AF, and would help balance the benefits of decreased fibrillatory burden against the morbidity of increased lesion load.

What is needed are medical systems, devices, components and methods that can be employed to more quickly, efficiently, cost-effectively, and accurately diagnose and treat patients who have AF using intravascular techniques, where cardiac or pulmonary vein tissue is likely to be ablated, and where accurate and enhanced EP mapping of the heart can be carried out. What is also needed are improved means and methods of acquiring intracardiac electrogram signals that quickly, reliably and accurately yield the precise locations and sources of cardiac rhythm disorders in a patient's heart. Doing so would enable cardiac ablation procedures to be carried out with greater speed, greater locational precision, lower risk to the patient, reduced cost, and higher rates of success in treating cardiac rhythm disorders such as AF. Still further, what is needed are lower cost methods of making EP mapping catheters, and improved designs, function, resolution, and reliability of EP mapping catheters.

### SUMMARY

In one embodiment, there is provided a Nitinol basket for an electrophysiological (EP) mapping catheter, where the catheter comprises a plurality of basket splines, each basket spline having a distalmost portion and a proximal end, and a distal tip uninterruptedly contiguous with the distalmost portions of the basket splines and formed from the same piece, slab or ingot comprising Nitinol as the splines, and wherein the basket splines and distal tip are cut and formed from a same single length of Nitinol tubing or a Nitinol hypotube, the respective distal portions of each of the Nitinol splines being continuous and contiguous with, and connected to, the Nitinol distal tip, each spline being configured to extend outwardly away from an imaginary central axis of the Nitinol basket and its proximal end and distal portion to form a curved shape therebetween when the Nitinol basket is in an undeformed and deployed state, the proximal ends of the splines being configured to be grouped adjacent or near one another and to extend together in a proximal direction for incorporation into or onto a distal end or distal portion of a catheter body, and further wherein the splines are configured to be spaced approximately equal distances apart from one another when the Nitinol basket is in an undeformed and deployed state, and still further wherein the splines collectively form a basket shape when the Nitinol basket is in an undeformed and deployed state.

In such an embodiment and others, the Nitinol basket may further comprise one or more of: (a) when the Nitinol basket is in an undeformed and deployed state, the basket shape is one of a spherical shape, a near-spherical shape, a tear-drop shape, a D-shape, a bulging asymmetric shape, and a shape where the splines are helically wound; (b) the single piece of Nitinol tubing prior to cutting has an initial inner diameter and an initial outer diameter, the initial inner diameter ranging between about 0.050 inches and about 0.070 inches, the initial outer diameter ranging between about 0.070 inches and about 0.080 inches; (c) the single piece of Nitinol tubing prior to cutting has an initial inner diameter and an initial outer diameter, the initial inner diameter ranging between about 0.1 inches and about 0.17 inches, the initial outer diameter ranging between about 0.1 inches and about 0.2 inches; (d) the proximal ends of the splines are configured collectively to form a plurality of adjoining and bunched splines or struts configured for insertion into or operable connection with the distal end or distal portion of the catheter body; (e) at least portions of the proximal ends of the splines comprise one or more of holes, recesses, shoulders, undercuts, and corners configured to promote attachment or securing of an adhesive or of a polymeric material or flowed, reflowed or reformed therethrough, therearound, or therein; (f) the basket comprises between 6 and 16 splines; (g) the basket comprises 8 splines, and the splines are configured to be spaced about 45 degrees apart from one another when the Nitinol basket is in an undeformed and deployed state; (h) the finished splines have opposing top and bottom surfaces, and the thickness between the top and bottom surfaces of the finished splines ranges between about 0.005 inches and about 0.008 inches; (i) the finished splines have opposing top and bottom surfaces, and the thickness between the top and bottom surfaces of the finished splines ranges between about 0.003 inches and about 0.012 inches; (j) the finished splines have opposing top and bottom surfaces, and one or more of the splines have variable thicknesses configured to induce flexing or bending of the splines at one or more predetermined locations; (k) the finished splines of the Nitinol basket have widths ranging between about 0.0150 inches and about 0.0180 inches; (I) the finished splines of the Nitinol basket have widths ranging between about 0.010 inches and about 0.025 inches; (m) the distal tip of the basket comprises an atraumatic shape or a polymeric covering or shield attached thereto, the atraumatic shape, covering or shield being configured to prevent or minimize injury to an interior surface of a patient's heart; (n) a flex circuit mounted on or attached to each or selected splines, each flex circuit comprising a plurality of electrodes mounted on or attached thereto; (o) a polymeric material flowed, reflowed or reformed onto at least portions of each spline to at least one of hold, secure and register or orient each flex circuit and its electrodes thereon or thereto; (p) the electrodes are unipolar electrodes or bipolar electrodes; and (q) after the splines and the distal tip have been cut from the Nitinol tubing or Nitinol hypotube, and before flex circuits, electrodes, and polymeric materials are flowed, reflowed or reformed onto or into the splines, the splines and basket are at least one of heat-set, quenched, media blasted, acid etched, and electropolished.

In another embodiment, there is provided a method of making a Nitinol basket for an electrophysiological (EP) mapping catheter, where the method comprises drawing or forming a Nitinol tube or hypotube from a single piece, slab or ingot comprising Nitinol, the drawn or formed Nitinol tube or hypotube having an initial inner diameter and an initial outer diameter; if required, cutting the drawn or formed Nitinol tube or hypotube to a desired length; cutting the drawn or formed Nitinol tube or hypotube of the desired length to form a plurality of basket splines and a distal tip, each cut basket spline having a distalmost portion and a proximal end, wherein the cut distal tip is uninterruptedly contiguous and continuous with, and connected to, the respective distalmost portions of the basket splines and is formed from the same Nitinol tube or hypotube as the splines; and wherein each spline of the resulting Nitinol basket is configured to extend outwardly away from an imaginary central axis of the Nitinol basket and its proximal end and distal portion to form a curved shape therebetween when the Nitinol basket is in an undeformed and deployed state, the proximal ends of the splines are configured to be grouped adjacent or near one another and to extend together in a proximal direction for incorporation into or onto a distal end or distal portion of a catheter body, and further wherein the splines are configured to be spaced approximately equal distances apart from one another when the Nitinol basket is in an undeformed and deployed state, and still further wherein the splines collectively form a basket shape when the Nitinol basket is in an undeformed and deployed state.

In such an embodiment and others, the method may further comprise one or more of: (a) shaping and treating the splines such that, when the finished Nitinol basket is in an undeformed and deployed state, the basket shape is one of a spherical shape, a near-spherical shape, a tear-drop shape, a D-shape, a bulging asymmetric shape, and a shape where the splines are helically wound; (b) drawing or forming the Nitinol tube or hypotube into a desired wall thickness comprising an initial outer diameter and a desired initial inner diameter; (c) the cutting is performed by one or more lasers; (d) heat-setting the cut plurality of basket splines and distal tip while disposed in a first die or tool configured to hold or retain the basket splines and distal tip in first predetermined positions and orientations during the heat-setting step; (e) quenching the heat-set and cut plurality of basket splines and distal tip while still disposed in the die or tool configured to hold or retain the basket splines and distal tip in the first predetermined positions and orientations during the quenching step; (f) heat-setting and quenching at least once more the previously heat-set and cut plurality of basket splines and distal tip; (g) heat-setting and quenching at least once more the previously heat-set and cut plurality of basket splines and distal tip while disposed in a second die or tool configured to hold or retain the basket splines and distal tip in second predetermined positions and orientations during the subsequent quenching step, the second die or tool being different from the first die or tool, and the second predetermined positions and orientations being different from the first predetermined positions and orientations; (h) the single piece, slab or ingot comprising Nitinol has super-elastic properties associated therewith; (i) super-elastic properties of the Nitinol basket are imparted thereto or enhanced therein by one or more of heat-setting steps and quenching steps; (j) an Austenite finish temperature of the Nitinol basket is less than the interior temperature of a live human patient, thereby to preserve or enhance super-elastic properties associated with the Nitinol basket; (k) after heat-setting and quenching steps, the Nitinol basket is subjected to one or more of media blasting, acid etching, and electropolishing; (I) imparting and forming an atraumatic shape, or providing an atraumatic covering or protective shield over, the distal tip, thereby preventing or minimizing injury to an interior surface of a patient's heart; (m) mounting or attaching a flex circuit on each spline, each flex circuit comprising a plurality of electrodes mounted on or attached thereto; and (n) flowing, reflowing or reforming a polymeric material onto or into at least portions of each spline to at least one of hold, secure and register or orient each flex circuit and its electrodes thereon or thereto.

Further embodiments are disclosed herein or will become apparent to those skilled in the art after having read and understood the claims, specification and drawings hereof.

### BRIEF DESCRIPTION OF THE DRAWINGS

Different aspects of the various embodiments will become apparent from the following specification, drawings and claims in which:

FIG. 1A shows an illustrative side perspective view of one embodiment of a Nitinol basket 10;

FIG. 1B shows a cross-sectional view of proximal end 16 and splines 12a-12h of basket 10 through section A-A′ of FIG. 1A;

FIG. 2 shows a top perspective view of one embodiment of Nitinol basket 10 similar to that illustrated in FIGS. 1A and 1B;

FIG. 3 shows a side perspective view of one embodiment of EP mapping catheter 1;

FIGS. 4A through 4D illustrate various details associated with the embodiment of basket 10 shown in FIGS. 1A through 3;

FIGS. 5A and 5B illustrate certain details according to one embodiment regarding the attachment of distal end 16 of basket 10 to catheter body 33 and distal shaft assembly 17 at junction 17/33;

FIG. 6 illustrates certain details according to another embodiment regarding the attachment of distal end 16 of basket 10 to catheter body 33 and distal shaft assembly 17 at junction 17/33;

FIG. 7 shows an end-on cross-sectional view, looking in the distal direction, of the exterior end of catheter handle 6;

FIGS. 8A and 8B show end and cross-sectional side views of one embodiment of a Nitinol tube or Nitinol hypotube 5;

FIG. 9 shows one embodiment of Nitinol tube or hypotube 5 after further laser cutting of tube 5 to form basket 10 in its initial stages of formation after step 105 of FIG. 10, and

FIG. 10 shows one embodiment of a method of making basket 10.

The drawings are not necessarily to scale. Like numbers refer to like parts or steps throughout the drawings.

### DETAILED DESCRIPTIONS OF SOME EMBODIMENTS

Disclosed herein are various embodiments of systems, devices, components and methods for diagnosing and treating cardiac rhythm disorders in a patient's heart using improved EP mapping and ablation catheters. Various embodiments described and disclosed herein also relate to systems, devices, components and methods for discovering with enhanced precision the location(s) of the source(s) of different types of cardiac rhythm disorders and irregularities. Such cardiac rhythm disorders and irregularities, include, but are not limited to, arrhythmias, atrial fibrillation (AF or A-fib), atrial tachycardia, atrial flutter, paroxysmal fibrillation, paroxysmal flutter, persistent fibrillation, ventricular fibrillation (V-fib), ventricular tachycardia, atrial tachycardia (A-tach), ventricular tachycardia (V-tach), supraventricular tachycardia (SVT), paroxysmal supraventricular tachycardia (PSVT), Wolff-Parkinson-White syndrome, bradycardia, sinus bradycardia, ectopic atrial bradycardia, junctional bradycardia, heart blocks, atrioventricular block, idioventricular rhythm, areas of fibrosis, breakthrough points, focus points, re-entry points, premature atrial contractions (PACs), premature ventricular contractions (PVCs), and other types of cardiac rhythm disorders and irregularities.

Also described herein is an EP mapping catheter having a Nitinol basket 10 that is capable of electrographically imaging a patient's atrium, left atrial appendage, portions of the pulmonary vein (PV) near the atrium, other heart chambers, and/or other internal organs at high or relatively high resolutions, and that can be manufactured at a lower cost, and with improved reliability, relative to high-electrode-density intra-cardiac EP mapping catheters of the prior art.

In the following description, for purposes of explanation, numerous specific details are set forth in order to provide a thorough understanding of example embodiments or aspects. It will be evident, however, to those skilled in the art that an example embodiment may be practiced without necessarily using all of the disclosed specific details, and that other embodiments not specifically or wholly disclosed are also contemplated and fall within the scope of the various inventions.

Problems that can and do occur using conventional basket catheters, such as spline bunching and poor electrode coverage, are described in considerable detail in the following publications: (a) “Basket-Type Catheters: Diagnostic Pitfalls Caused by Deformation and Limited Coverage” to Oesterlein et al., BioMed Research International, Volume 2016, Article ID 5340574 (“the Oesterlein publication”); (b) “Practical Considerations of Mapping Persistent Atrial Fibrillation With Whole-Chamber Basket Catheters” to Laughner et al., JACC: Clinical Electrophysiology, Volume 2, Issue 1, February 2016, Pages 55-65 (“the first Laughner publication”); and (c) “Atrial Mapping With Basket Catheters— A Basket Case?” To Hummel et al., JACC: Clinical Electrophysiology, Volume 2, Issue 1, February 2016, Pages 66-68 (“the second Laughner publication”).

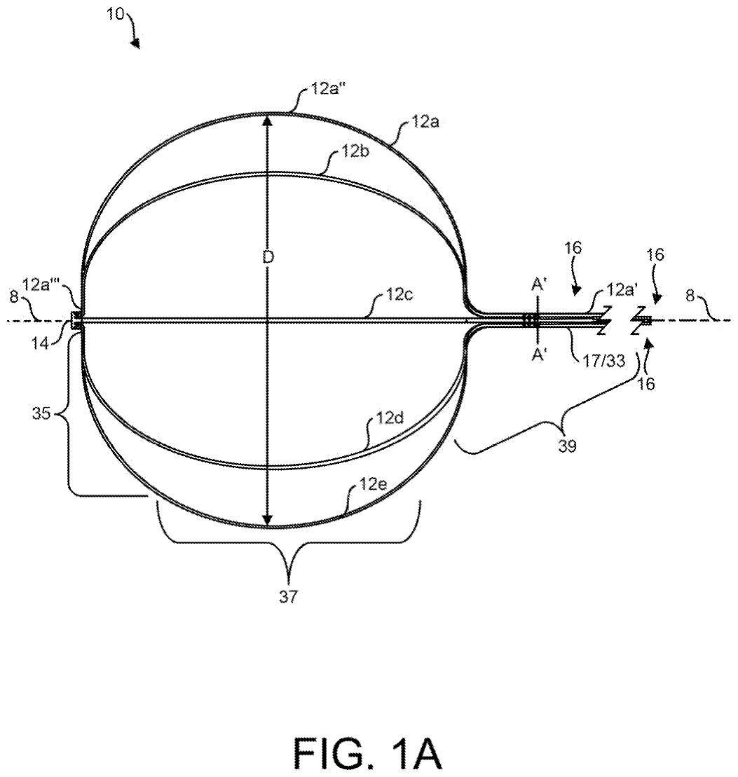

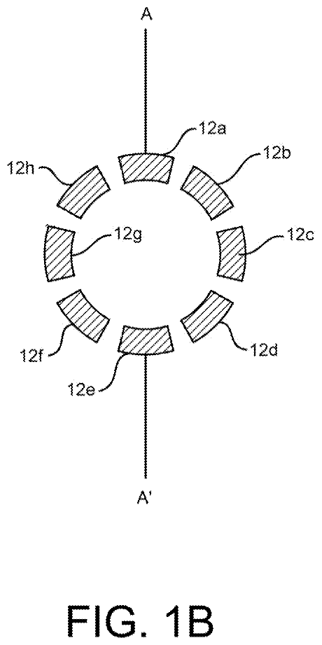

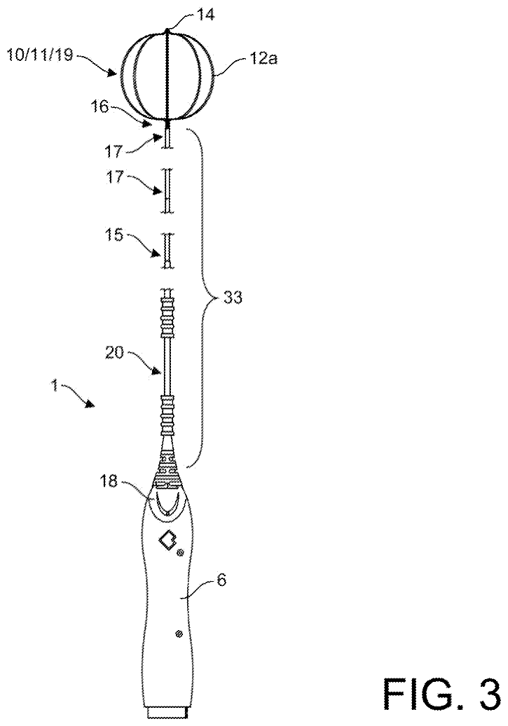

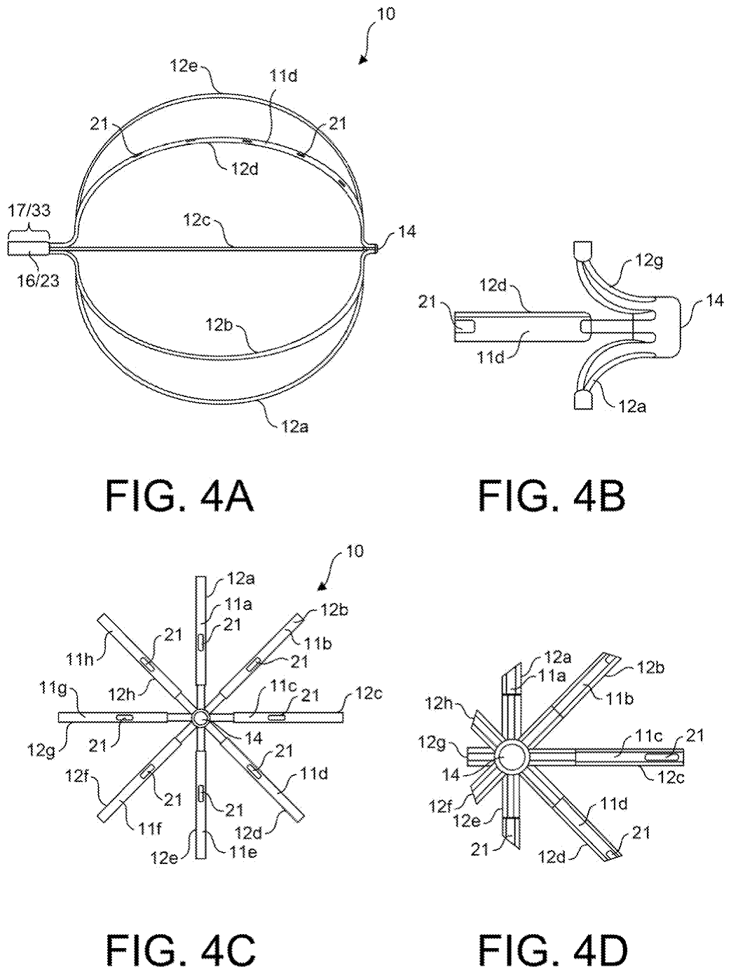

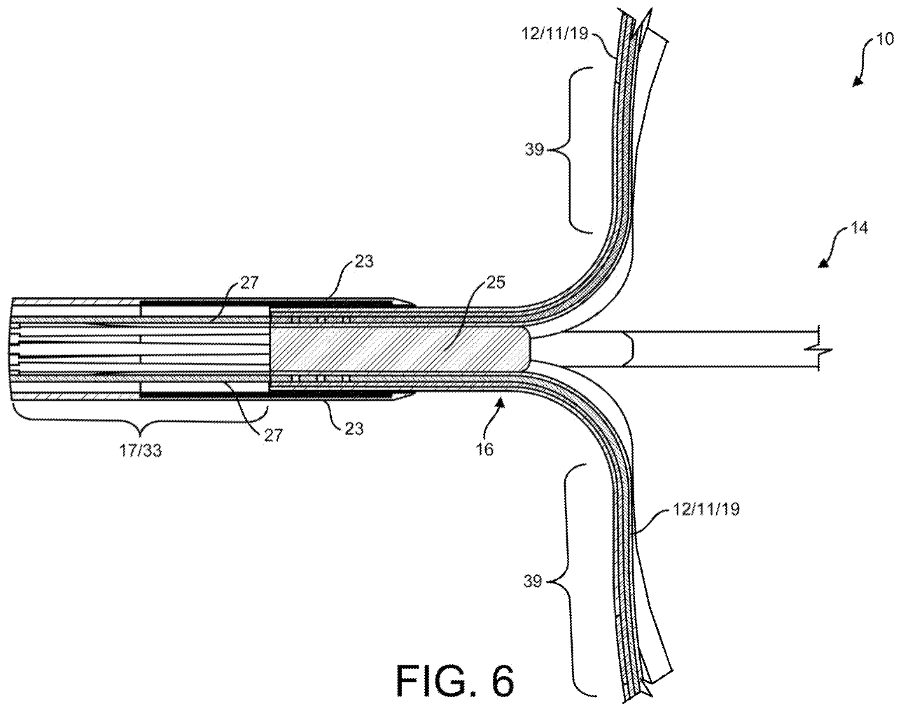

Referring now to FIG. 1A, there is shown an illustrative side perspective view of one embodiment of a Nitinol basket 10, which is shown apart from other portions of a complete EP mapping catheter 1 (see, for example FIG. 3). As shown in the illustrated embodiment of FIG. 1A, Nitinol basket 10 comprises a plurality of eight basket splines 12a through 12h (splines 12f, 12g and 12h being hidden by other splines). Each basket spline has a distalmost portion 35 and a proximal end 16. Distal tip 14 is uninterruptedly contiguous and continuous with distalmost portions 35 of basket splines 12a-12h and has been formed from the same piece, slab or ingot comprising Nitinol as the splines (more about which is said below). That is, basket splines 12a-12h and distal tip 14 are cut and formed from the same single piece or length of Nitinol tubing or a Nitinol hypotube, where the respective distal portions 35 of each of the Nitinol splines 12a-12h are continuous and contiguous with, and connected to, Nitinol distal tip 14. As shown in FIG. 1A, each spline is configured to extend outwardly away from imaginary central axis 8 of the Nitinol basket and its proximal end portion 39 and distal portion 35 to form a curved shape therebetween when the Nitinol basket is in an undeformed and deployed state. In many embodiments, Nitinol basket 10 is further configured to be collapsed into a narrowed shape for containment within a sheath, introducer or other containment and delivery device for delivery inside a patient's atrium (or other heart chamber or internal organ) and then deployment or expansion therewithin, as is well known by those skilled in the art. The proximal ends or portions 16 of the splines 12 are configured to be grouped adjacent or near one another and to extend together in a proximal direction for incorporation into or onto a distal end or distal portion 17 of a catheter body (see, for example, portions 17 of catheter 1 in FIG. 4, or molding 23 and polymeric material 25 in FIGS. 6A, 68 and 7). As shown in FIG. 1A, splines 12 are configured to be spaced approximately equal distances apart from one another when the Nitinol basket is in an undeformed and deployed state. Splines 12 collectively form a basket shape when the Nitinol basket 10 is in an undeformed and deployed state (as shown in FIG. 1A). In the embodiment shown in FIG. 1A, medial portions 37 of splines 12 are bowed or curved outwardly away from imaginary axis 8 when the Nitinol basket 10 is in an undeformed and deployed state.

Continuing to refer to FIG. 1A, and by way of illustrative example, spline 12a is shown to have proximal portion 12a′, medial portion 12a″, and distal portion 12a′″ located adjacent to and contiguous with distal tip 14.

FIG. 1B shows a cross-sectional view of proximal end 16 and splines 12a-12h of basket 10 through section A-A′ of FIG. 1A. As shown in FIG. 1B, in one embodiment, and at proximal location 16 or other locations, splines 12a-12h can each have a finished thickness (after media blasting, electropolishing, etc., more about which is said below) of about 0.0065″+/−0.0010″, and a width of about 0.0165″+/−0.0010 inches. Other more narrow or greater thicknesses and widths of splines are 12 are also contemplated, as described elsewhere herein, which in some embodiments depends at least partially upon the degree or amount of flexibility or stiffness desired in basket 10.

In some embodiments, however, the single piece of Nitinol tubing prior to cutting from which basket 10 is formed has an initial inner diameter and an initial outer diameter, where the initial inner diameter ranges between about 0.050 inches and about 0.070 inches, and the initial outer diameter ranges between about 0.070 inches and about 0.080 inches. In other embodiments where EP mapping catheter 1 has an inner lumen large enough to accept an ablation catheter therein and therethrough, the single piece of Nitinol tubing prior to cutting from which Nintinol basket 10 is formed has an initial inner diameter and an initial outer diameter, where the initial inner diameter ranges between about 0.1 inches and about 0.17 inches, and the initial outer diameter ranges between about 0.1 inches and about 0.2 inches.

As further shown in FIGS. 1A and 1B, the proximal ends or portions 16 of splines 12 of Nitinol basket 10 are configured collectively to form a plurality of adjoining and bunched struts configured for insertion into or operable connection to or with the distal end or distal portion 17 of catheter body 33.

According to various embodiments, when Nitinol basket 10 is in an undeformed and deployed state, the basket shape of Nitinol basket 10 may be one or a combination of a spherical shape, a near-spherical shape, a tear-drop shape, a D-shape, a bulging asymmetric shape, and/or a shape where splines 12 are helically wound. Other suitable shapes for basket 10 are also contemplated.

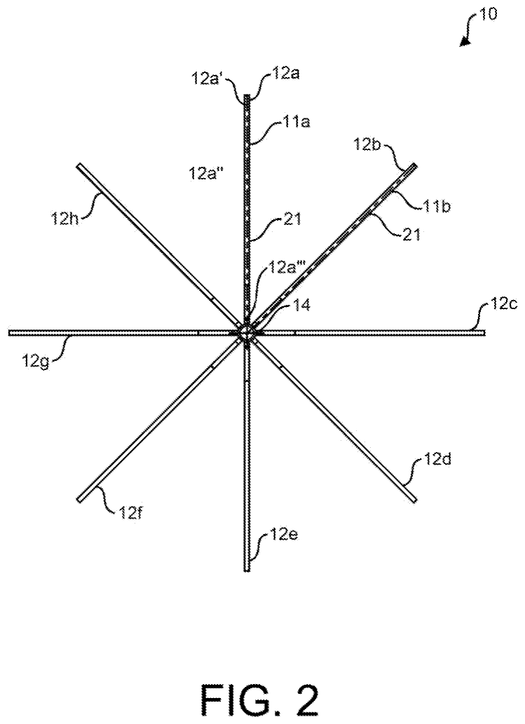

Referring now to FIG. 2, there is shown a top perspective view of one embodiment of Nitinol basket 10 similar to that illustrated in FIGS. 1A and 1B, where basket 10 comprises 8 splines 12a-12h, and splines 12a-12f are configured to be spaced about 45 degrees apart from one another when the Nitinol basket is in an undeformed and deployed state. Other spacings of splines 12 are contemplated, including irregular spacings. In FIG. 2, splines 12a and 12b are shown with flex circuits 11a and 11b mounted thereon or attached thereto. Flex circuits 21 can be mounted on or attached to each or selected splines 12, each flex circuit 21 comprising a plurality of electrodes mounted on or attached thereto. For purposes of illustration, and in the embodiment shown in FIG. 2, flex circuits 11a and 11b are shown disposed on splines 12a and 12b, which each contain 8 separate electrodes 22, which are configured to be disposed along the respective lengths of splines 12a and 12b such that electrodes 22 will engage intra-cardiac sidewalls and sense electrical activity therein or thereon. See, for example, U.S. Pat. No. 10,925,542 to Jung et al. entitled “Multi-Electrode Mapping Catheter with Continuous Flex Circuit,” which discloses certain aspects of incorporating or using flex circuits in EP mapping catheters, and which is hereby incorporated by reference herein, in its entirety.

Referring now to FIGS. 1A, 1B, 2, and 5A-6, electrodes 21 shown and displayed in the illustrated embodiments are unipolar electrodes. Bipolar and tripolar electrodes, however, are also contemplated for use in conjunction with basket 10, as are body surface electrodes.

In some embodiments, at least portions of splines 12a-12h can be configured to include or comprise one or more of holes, recesses, shoulders, undercuts, corners or other suitable structural features so that the attachment or securing of flex circuits 11a-11h along, atop or otherwise situated on or to splines 12a-12h can be facilitated using an adhesive, or a polymeric material such as PEBAX® (or Polyether block amide or PEBA, which is a thermoplastic elastomer or TPE), which can be disposed upon, flowed, reflowed or reformed through, around, or in portions of splines 12a-12h. In the embodiments shown in FIGS. 1A, 1B, 2, and 5A-6, splines 12a-12h contain a total of 64 electrodes, or 8 electrodes 21 on each spline 12.

Continuing to refer to FIG. 2, in its various embodiments basket 10 can comprise between 6 and 16 splines, or any other suitable number of splines. Additionally, although the embodiments of basket 10 shown in FIGS. 1A, 1B, 2, and 5A-6 are configured to include 8 splines and 8 electrodes on each spline, other numbers of splines and numbers of electrodes disposed on such splines are also contemplated, as those skilled in the art will understand. For example, in an embodiment where basket 10 comprises 12 splines, with 8 electrodes disposed on each spline, basket 10 comprises a total of 96 electrodes. in an embodiment where basket 10 comprises 16 splines, with 8 electrodes disposed on each spline, basket 10 comprises a total of 128 electrodes. Other numbers of electrodes 21 per spline 12 are also contemplated, including, but not limited to, 4, 6, 8, 10, 12, 14, and 16 electrodes 21 per spline 12.

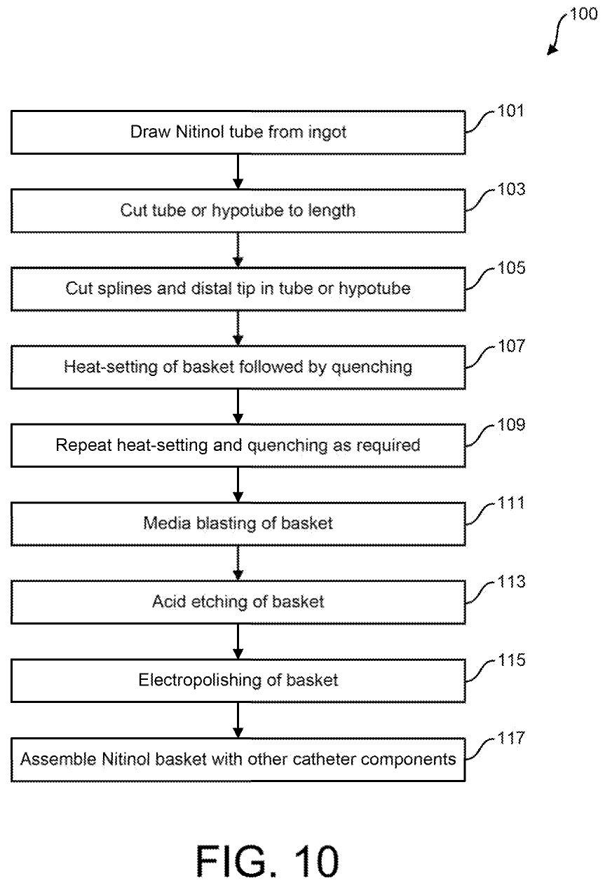

In some embodiments, and with continued reference to FIGS. 1A, 1B, 2, and 5A-6, finished splines 12a-12h have opposing top and bottom surfaces, and the thickness between the top and bottom surfaces of the finished splines ranges between about 0.005 inches and about 0.008 inches. Finished splines 12a-12h result from carrying out steps 101-115 shown in FIG. 10 (more about which is said below). In other embodiments, finished splines 12a-12h have opposing top and bottom surfaces, and the thickness between the top and bottom surfaces of the finished splines ranges between about 0.003 inches and about 0.012 inches. In still further embodiments, finished splines 12a-12h have opposing top and bottom surfaces, and one or more of splines 12 have variable thicknesses configured to induce or facilitate preferential flexing or bending of the splines at one or more predetermined locations, and in one embodiment may also have widths ranging between about 0.0150 inches and about 0.0180 inches, and in another embodiment may range between about 0.010 inches and about 0.025 inches.

Referring now to FIGS. 1A, 2, 3, 4A, 4B, 4C, 4D, an 9, there is shown distal tip 14 of basket 10, which can comprise or include an atraumatic shape, and/or a polymeric covering or shield attached thereto, such atraumatic shape, covering or shield being configured to prevent or minimize injury to an interior surface of a patient's heart when basket 10 is deployed therein.

In addition, after splines 12 and distal tip 14 have been cut from Nitinol tubing or a Nitinol hypotube, and before flex circuits 11, electrodes 21, and adhesives are applied thereto, or polymeric materials are flowed, reflowed or reformed onto or into splines 11, splines 11 and basket 10 are at least one of heat-set, quenched, media blasted, acid etched, and electropolished, as described in further detail below.

FIG. 3 shows a side perspective view of one embodiment of EP mapping catheter 1, which as shown comprises catheter handle 6, molded handle plug 18, introducer assembly 20, proximal shaft assembly 15, distal shaft assembly 17, basket 10, flex circuits 11, splines 12a-12h, polymeric moldings and coverings 19 on flex circuits 11 and splines 12, proximal end 16 of basket 10 attached to the distal end of distal shaft assembly 17, tip or atraumatic tip 14 of basket 10, and catheter body 33 comprising introducer assembly 20, proximal shaft assembly 15, and distal shaft assembly 17.

FIGS. 4A through 4D illustrate various details associated with the embodiment of basket 10 shown in FIGS. 1A through 3, including splines 12a-12h, tip 14, electrodes 21, flex circuits 11, and proximal end 16 of basket 10 attached to molding and securing means 23 disposed around proximal end 16 of basket 10, thereby to mechanically attach basket 10 to catheter body 33 and distal shaft assembly 17.

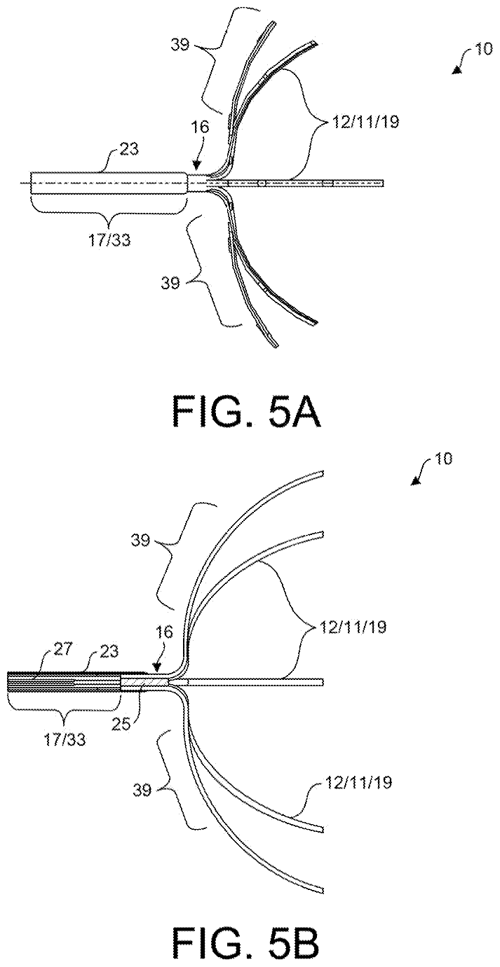

FIGS. 5A, 5B and 6 illustrate certain details regarding the attachment of proximal end 16 of basket 10 to catheter body 33 and distal shaft assembly 17 according to different views and embodiments thereof. Note that different embodiments and views of junction 17/33 between distal shaft assembly 17 and catheter body 33 are shown in FIGS. 1A, 4A, 5A, 5B and 6. Overmolding 23 can be used to secure at least partially distal end 16 to catheter body 33 and distal shaft assembly 17 at or over junction 17/33, as can polymeric moldings 25 and 27, which preferably comprise PEBAX or any other suitable polymeric material capable of being flowed, reflowed, or reformed around basket and catheter components such as flex circuits 11, splines 12, and/or other components of catheter 1 and basket 10.

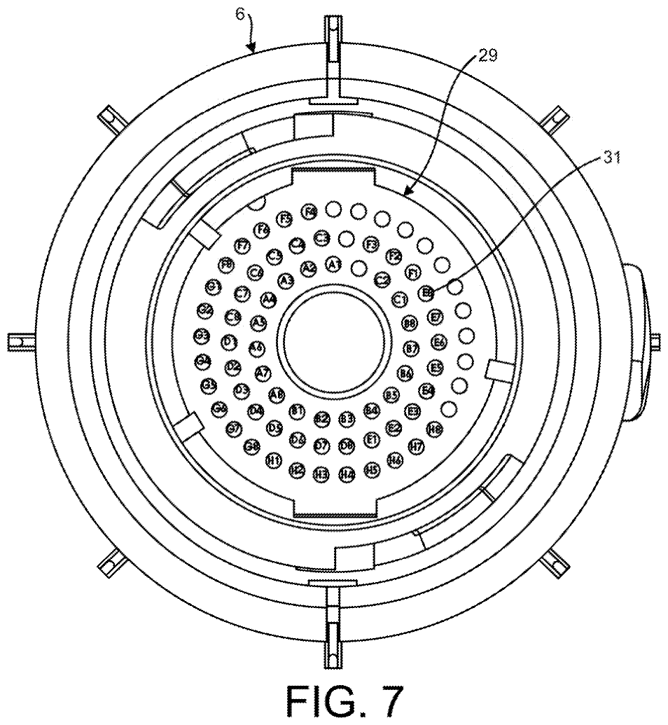

Turning now to FIG. 7, there is shown an end-on cross-sectional view, looking in the distal direction, of a portion of the exterior distal end of catheter handle 6, which as shown comprises one embodiment of a receiver socket 29 comprising electrical connection recesses and corresponding electrical contacts 31 configured to receive corresponding electrical connection pins located in an external cable operably connected to, in one embodiment, an amplifier, which in turn is operably connected to an external computing device configured to process the incoming signals provided by catheter 1. Not shown in the drawings are the proximal ends of flex circuits 11, which in one embodiment extend continuously or in sections from basket 10 to handle 6 for electrical connection to socket 29. As shown in the embodiment of FIG. 7, receiver socket 29 comprises 78 electrical connection recesses and corresponding electrical contacts 31, although in one embodiment, where 8 electrodes 21 are disposed on each of 8 splines 12, only 64 of contacts 31 need actually be employed. Of course other numbers of electrical connections, and other types of electrical connections between handle 6 and basket 10, are contemplated.

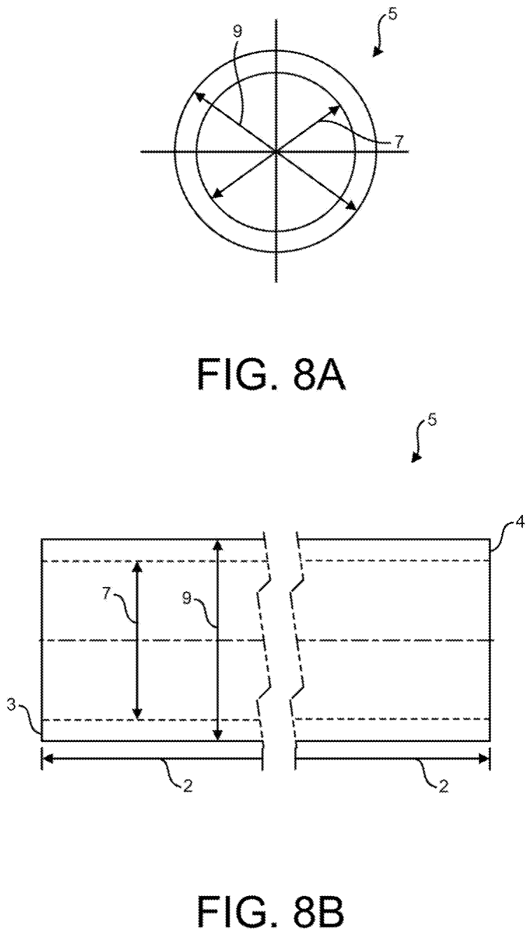

FIGS. 8A and 8B show end and cross-sectional side views of one embodiment of a Nitinol tube or Nitinol hypotube 5 comprising inner diameter 7, outer diameter 9, where tube or hypotube 5 comprises distal end 3 and proximal end 4. Tube or hypotube 5 shown in FIGS. 8A and 8B is the result of having been drawn from a single piece or ingot of Nitinol, and then cut to suitable length 2. Inner and outer diameters 7 and 9 shown in FIGS. 8A and 8B are those associated with Nitinol tube or hypotube 5 following metal or metal alloy drawing and cutting steps 101 and 103 outlined in basket fabrication, making or manufacturing method 100 of FIG. 10.

As described above, and in the embodiment shown in FIGS. 8A and 8B, the single piece of Nitinol tube or hypotube 5, prior to and just after cutting, has an initial inner diameter and an initial outer diameter, where the initial inner diameter ranges between about 0.050 inches and about 0.070 inches, and the initial outer diameter ranges between about 0.070 inches and about 0.080 inches. These dimensions change and diminish as tube or hypotube 5 is further machined and process according to, in one embodiment, method 100 of FIG. 10, as further steps 105 through 117 are carried out. Further, and as described above, other embodiments are contemplated where EP mapping catheter 1 and basket 10 have inner lumens large enough to accept an ablation catheter therein and therethrough, and the single piece of Nitinol tubing 5 prior to cutting from which Nintinol basket 10 is formed has an initial inner diameter and an initial outer diameter, where the initial inner diameter ranges between about 0.1 inches and about 0.17 inches, and the initial outer diameter ranges between about 0.1 inches and about 0.2 inches. Other dimensions of Nitinol tubing 5 are of course contemplated.

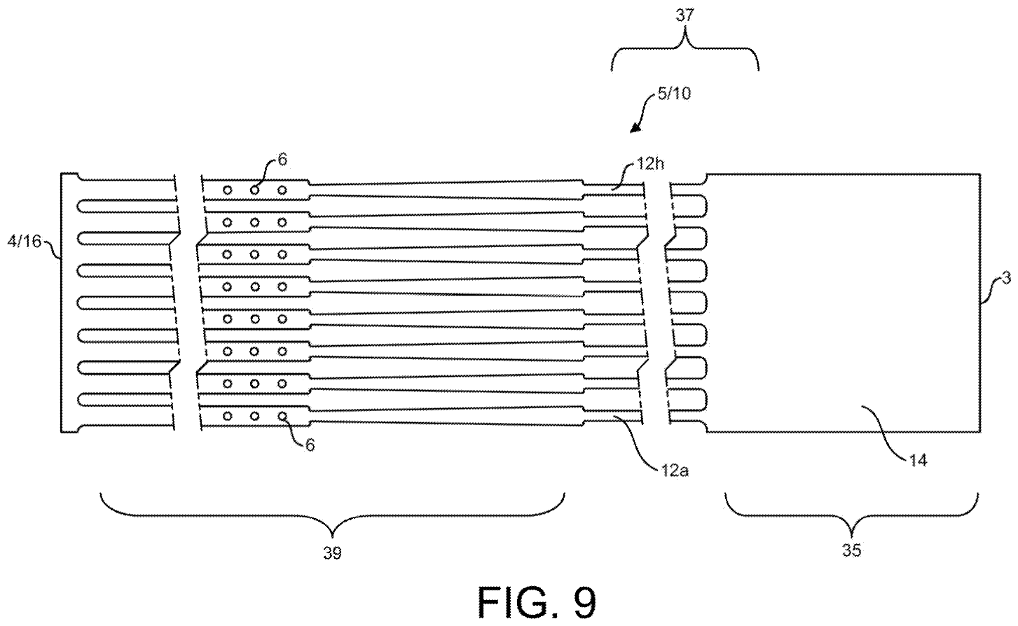

FIG. 9 shows one embodiment of Nitinol tube or hypotube 5 after further laser cutting of tube 5 to form basket 10 in its initial stages of formation after step 105 of FIG. 10 (cutting splines 12 in tube or hypotube 5). Note that proto-basket 10 and tube or hypotube 5 shown in FIG. 9 actually form a tube-shaped member or device after laser cutting, and that proto-basket 10 and tube or hypotube 5 shown in FIG. 9 is shown in a plan or a flattened view for purposes of illustration and visual convenience only. Further, distal end 16 of basket 10 in FIG. 9 is subsequently cut across the proximal base of tube or hypotube 5 so that the proximal ends of splines 12 are separated from one another. Note further that in FIG. 9 distal tip 14 is uninterruptedly contiguous with the distalmost portions of basket splines 12 and has been formed from the same piece, slab or ingot comprising Nitinol as splines 12. That is, basket splines 12 and distal tip 14 are cut and formed from the same single length of Nitinol tubing or a Nitinol hypotube 5, and the respective distal portions of each of Nitinol splines 12 is continuous and contiguous with, and connected to, Nitinol distal tip 14.

Methods of cutting tube or hypotube 5 include not only the use of suitable laser cutting methods, but also of mechanical and chemical cutting or shaping methods, including but not limited to mechanical cutting or sawing, abrading, grinding, chemical dissolution or etching, and other suitable means and methods of forming cut-outs and other feature in tube or hypotube 5.

Turning now to FIG. 10, there is shown one embodiment of a method of making basket 10, which includes steps 101 through 117. Method 100 illustrated in FIG. 10 is not intended to be comprehensive in all respects, and additional steps other than those shown explicitly in FIG. 10 are contemplated. Additionally, in various embodiments some steps shown in FIG. 10 may be replaced by other steps not shown explicitly shown therein, and in other embodiments some steps shown in FIG. 10 may not be included in method 100.

As shown in FIG. 10, one embodiment of method 10 of forming or making Nitinol basket 10 comprises the following steps: Step 101: Draw Nitinol tube or hypotube 5 from an ingot or other single piece or slab of Nitinol; Step 103: Cut Nitinol tube or hypotube 5 to length; Step 105: Cut splines 12 and distal tip 14 from tube or hypotube 5 using, e.g., laser cutting, mechanical, and/or chemical means or methods; Step 107; Heat-setting of partially formed basket 10 from Step 105, e.g., in a tool or die, followed by quenching; Step 109: Repeat heat-setting and quenching as required or desired; Step 111: media blasting of basket 10 using, e.g., plastic beads or sand; Step 113: acid etching of media-blasted basket 10; Step 115: electropolishing of basket 10; Step 117: Assemble Nitinol basket 10 with other components of catheter 1.

Continuing to refer to FIG. 10 and also to FIG. 9, in some embodiments method 100 begins with an ingot, slab or single piece of Nitinol metal (a nickel and titanium alloy), which is drawn into tube or hypotube 5. Once drawn into desired tubing diameters and wall thicknesses, tube or hypotube 6 is cut into discrete suitable lengths. The individual splines or arms 12 of basket 10 are then carefully cut in the wall of tube or hypotube 5. Laser cutting is the preferred method of cutting due to the small feature sizes of tube or hypotube 5 and splines 12. Care must be used in selecting the laser type and parameters so as to not impart excessive amounts of heat effect to uncut portions of tube or hypotube 5.

Once splines 12, distal tip 14, and additional features are cut into Nitinol tube or hypotube 5, a heat-setting process is performed with in some embodiments can comprise submerging the proto-basket 10 into a material forming a heated medium (e.g., a suitable molten salt medium), followed by a rapid quenching step. The first step of heat-setting is placing the laser cut Nitinol basket 10 in a fixture, tool or die that expands splines 12 out into a basket shape. The expanded Nitinol basket 10 and its fixture are then submerged into a heated medium bath (e.g., fluidized sand or aluminum oxide bath, molten salt bath). Typical temperatures of the heated medium range from 400°-600° C., but may be higher or lower depending on the material, tooling, and desired final properties. Similarly, time in the heated bath can range from a few seconds to several minutes depending on the density of the material, the tooling, and the desired process outcome. Once removed from the heated medium bath, basket 10 and the tooling are quenched in either liquid or air to rapidly reduce the temperature. The time of submergence in the heated bath and quench environment have an effect on the final properties. The heat-setting process may be performed multiple times in order to achieve the final desired shape, mechanical, and thermal properties. For example, the tool used in the first heat step cycle may only expand the laser cut nitinol splines out to 50% of the final shape. After the first heat-setting cycle the material might be loaded into a second tool that expands the nitinol splines out the rest of the way to 100% of the desired basket dimensions (followed by a quenching step).

Superelasticity, the ability to withstand higher than strains without deformation, is a desirable property of the finished nitinol basket 10. As such, selecting nitinol hypotube with certain initial phase transition values and either maintaining or altering those values as desired through the heat-setting processes important to the final form. For a mapping basket catheter, it is important that the Af temperature (Austenite finish) temperature be below the interior body temperature of a patient in order to realize the super-elastic properties during use.

After the shape setting process is complete, post-processing steps including media blasting, acid etching, and electropolishing are performed to remove residual oxide layers from the heat-setting process, surface defects like cracks and notches, and free nickel from the surface. Once these surface finish processes are complete, the nitinol basket is ready for assembly with the additional components of the catheter.

As described above, the use of a nitinol shape memory or super-elastic element for a particular application generally requires the setting of a custom shape in a piece of nitinol. The process required to set the shape is similar whether beginning with nitinol in the form of wire, strip, sheet, tubing, rod or bar. Shape setting (or training) is accomplished by constraining the nitinol element on a mandrel or fixture of the desired shape and applying an appropriate heat treatment. The heat treatment methods used to set shapes in both shape memory and super-elastic forms of nitinol are similar.

The heat treatment parameters chosen to set both the shape and the properties of the part are critical, and usually need to be determined experimentally for each desired part's requirements. In general, temperatures as low as 400 deg. C. and times as short as 1-2 minutes can set the shape, but generally one uses a temperature closer to 500 deg. C. and times over 5 minutes. Rapid cooling of some form is preferred via a water quench or rapid air cool (if both the parts and the fixture are small). Higher heat treatment times and temperatures will increase the actuation temperature of the part and often gives a sharper thermal response (in the case of shape memory elements). However, there is usually a concurrent drop either in peak force (for shape memory elements) or in plateau stresses (for super-elastic elements). There is also an accompanying decrease in the ability of the Nitinol element to resist permanent deformation.

In some embodiments, splines 12 disclosed and described herein comprise a biocompatible shape memory alloy (e.g., nickel titanium, or Nitinol), and have been treated and configured during the process of manufacturing splines 12 and catheter 1 such that splines 12 assume an outwardly curving shape as they are progressively exposed by the withdrawal of a sheath or introducer, or as splines 12 are advanced from a distal end of catheter 1.

Nitinol is a metal alloy of nickel and titanium, where the two elements are typically present in roughly equal atomic percentages, e.g., Nitinol 55, Nitinol 60. The properties of the Nitinol or other suitable shape memory alloy employed in tube or hypotube 5 and splines 12 are particular to the precise composition of the alloy used and its processing, and in some embodiments exhibit shape memory effect (SME) and superelasticity (SE; also called pseudoelasticity, PE). Nitinol is highly biocompatible, and has properties suitable for use in medical devices inserted or implanted within the human body. Due to Nitinol's unique properties, Nitinol finds application in catheters, stents, and super elastic needles.

In embodiments where the shape memory alloy selected for use in catheter 1 and basket 10 is Nitinol, tight compositional control of the Nitinol is required during the manufacturing process due to the high reactivity of titanium. By way of example, melting methods of the Nitinol employed to form tube or hypotube 5, splines 12 and distal tip 14 may include vacuum arc remelting (VAR) or vacuum induction melting (VIM). High vacuums may be required during a Nitinol spline manufacturing process. Alternatives to VAR and VIM include, but are not limited to, plasma arc melting, induction skull melting, and e-beam melting. Physical vapor deposition may also be employed. Some methods of working Nitinol for use in splines 12 include, but are not limited to, grinding, abrasive cutting, electrical discharge machining (EDM), and laser cutting. Heat treating of Nitinol employed in splines 12 can include varying aging time and temperature controls to obtain a desired Ni-rich phase and transformation temperature of splines 12, and thus control how much nickel resides in the resulting NiTi lattice. With respect to catheter 1, basket 10 and splines 12 thereof, Nitinol is worked, treated and formed so that it will consistently and reliably behave and assume one or more of the various configurations shown and described herein as mapping electrode assembly or basket 10 is progressively deployed from a distal end of catheter 1.

In alternative embodiments, splines 12 and distal tip 14 may comprise a biocompatible material having shape memory characteristics and attributes, but are not formed of Nitinol or other shape memory alloys (or at least are not formed primarily or solely of one or more shape memory alloys). By way of non-limiting example, in such alternative embodiments splines 12 and distal tip 14 are formed of biocompatible shape memory materials such as shape-memory polymers, laminated 3D printed splines comprising shape memory materials, shape memory composites, and/or shape memory hybrids.

Various aspects and features of basket 10 and catheter 1 described and disclosed herein may be modified to include elements, features and steps described and disclosed in one or more of the '183, '697, '069, '637, '873, '368, and '594 patent applications described hereinabove in the “Related Applications” section. The various systems, devices, components and methods described and disclosed herein may also be adapted and configured for use in EP mapping applications in the heart's atria, ventricles, pulmonary vein or arteries, or other portions of the heart, and may further be configured for use in EP mapping in organs other than those involving the interior of a patient's heart or the pulmonary veins or arteries. These alternative applications include, but are not limited to, EP mapping and diagnosis, or other forms, means or methods of electrically sensing, a patient's stomach, colon, esophagus, veins, arteries, aorta, or any other suitable portion of a patient's body. The various embodiments further include within their scope methods of implanting, using and making the catheters described hereinabove.

What have been described above are examples and embodiments of the devices and methods described and disclosed herein. It is, of course, not possible to describe every conceivable combination of components or methodologies for purposes of describing the invention, but one of ordinary skill in the art will recognize that many further combinations and permutations of the devices and methods described and disclosed herein are possible. Accordingly, the devices and methods described and disclosed herein are intended to embrace all such alterations, modifications and variations that fall within the scope of the appended claims. In the claims, unless otherwise indicated, the article “a” is to refer to “one or more than one.”

The foregoing outlines features of several embodiments so that those skilled in the art may better understand the detailed description set forth herein. Those skilled in the art will now understand that many different permutations, combinations and variations of basket 10 and method 100 fall within the scope of the various embodiments. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

After having read and understood the present specification, those skilled in the art will now understand and appreciate that the various embodiments described herein provide solutions to long-standing problems, both in the use of electrophysiological mapping systems and in the use of cardiac ablation systems.

We claim:

1. A Nitinol basket for an electrophysiological (EP) mapping catheter, comprising:
a plurality of basket splines, each basket spline having a distalmost portion and a proximal end, and
a distal tip uninterruptedly contiguous with the distalmost portions of to the basket splines and formed from the same piece, slab or ingot comprising Nitinol as the splines;
wherein the basket splines and distal tip are cut and formed from a same single length of Nitinol tubing or a Nitinol hypotube, the respective distal portions of each of the Nitinol splines being continuous and contiguous with, and connected to, the Nitinol distal tip, each spline being configured to extend outwardly away from an imaginary central axis of the Nitinol basket and its proximal end and distal portion to form a curved shape therebetween when the Nitinol basket is in an undeformed and deployed state, the proximal ends of the splines being configured to be grouped adjacent or near one another and to extend together in a proximal direction for incorporation into or onto a distal end or distal portion of a catheter body, and further wherein the splines are configured to be spaced approximately equal distances apart from one another when the Nitinol basket is in an undeformed and deployed state, and still further wherein the splines collectively form a basket shape when the Nitinol basket is in an undeformed and deployed state.

2. The Nitinol basket of claim 1, wherein, when the Nitinol basket is in an undeformed and deployed state, the basket shape is one of a spherical shape, a near-spherical shape, a tear-drop shape, a D-shape, a bulging asymmetric shape, and a shape where the splines are helically wound.

3. The Nitinol basket of claim 1, wherein the single piece of Nitinol tubing prior to cutting has an initial inner diameter and an initial outer diameter, the initial inner diameter ranging between about 0.050 inches and about 0.070 inches, the initial outer diameter ranging between about 0.070 inches and about 0.080 inches.

4. The Nitinol basket of claim 1, wherein the single piece of Nitinol tubing prior to cutting has an initial inner diameter and an initial outer diameter, the initial inner diameter ranging between about 0.1 inches and about 0.17 inches, the initial outer diameter ranging between about 0.1 inches and about 0.2 inches.

5. The Nitinol basket of claim 1, wherein the proximal ends of the splines are configured collectively to form a plurality of adjoining and bunched struts or splines configured for insertion into or operable connection with the distal end or distal portion of the catheter body.

6. The Nitinol basket of claim 1, wherein at least portions of the proximal ends of the splines comprise one or more of holes, recesses, shoulders, undercuts, and corners configured to promote attachment or securing of an adhesive or of a polymeric material or flowed, reflowed or reformed therethrough, therearound, or therein.

7. The Nitinol basket of claim 1, wherein the basket comprises between 6 and 16 splines.

8. The Nitinol basket of claim 5, wherein the basket comprises 8 splines, and the splines are configured to be spaced about 45 degrees apart from one another when the Nitinol basket is in an undeformed and deployed state.

9. The Nitinol basket of claim 1, wherein the finished splines have opposing top and bottom surfaces, and the thickness between the top and bottom surfaces of the finished splines ranges between about 0.005 inches and about 0.008 inches.

10. The Nitinol basket of claim 1, wherein the finished splines have opposing top and bottom surfaces, and the thickness between the top and bottom surfaces of the finished splines ranges between about 0.003 inches and about 0.012 inches.

11. The Nitinol basket of claim 1, wherein the finished splines have opposing top and bottom surfaces, and one or more of the splines have variable thicknesses configured to induce flexing or bending of the splines at one or more predetermined locations.

12. The Nitinol basket of claim 1, wherein the finished splines of the Nitinol basket have widths ranging between about 0.0150 inches and about 0.0180 inches.

13. The Nitinol basket of claim 1, wherein the finished splines of the Nitinol basket have widths ranging between about 0.010 inches and about 0.025 inches.

14. The Nitinol basket of claim 1, wherein the distal tip of the basket comprises an atraumatic shape or a polymeric covering or shield attached thereto, the atraumatic shape, covering or shield being configured to prevent or minimize injury to an interior surface of a patient's heart.

15. The Nitinol basket of claim 1, further comprising a flex circuit mounted on or attached to each spline, each flex circuit comprising a plurality of electrodes mounted on or attached thereto.

16. The Nitinol basket of claim 15, further comprising a polymeric material flowed, reflowed or reformed onto at least portions of each spline to at least one of hold, secure and register or orient each flex circuit and its electrodes thereon or thereto.

17. The Nitinol basket of claim 15, wherein the electrodes are one or a combination of unipolar and bipolar electrodes.

18. The Nitinol basket of claim 1, wherein after the splines and the distal tip have been cut from the Nitinol tubing or Nitinol hypotube, and before flex circuits, electrodes, and polymeric materials are flowed, reflowed or reformed onto or into the splines, the splines and basket are at least one of heat-set, quenched, media blasted, acid etched, and electropolished.

19. A method of making a Nitinol basket for an electrophysiological (EP) mapping catheter, comprising:
drawing or forming a Nitinol tube or hypotube from a single piece, slab or ingot comprising Nitinol, the drawn or formed Nitinol tube or hypotube having an initial inner diameter and an initial outer diameter;
if required, cutting the drawn or formed Nitinol tube or hypotube to a desired length;
cutting the drawn or formed Nitinol tube or hypotube of the desired length to form a plurality of basket splines and a distal tip, each cut basket spline having a distalmost portion and a proximal end, wherein the cut distal tip is uninterruptedly contiguous and continuous with, and connected to, the respective distalmost portions of the basket splines and is formed from the same Nitinol tube or hypotube as the splines;
wherein each spline of the resulting Nitinol basket is configured to extend outwardly away from an imaginary central axis of the Nitinol basket and its proximal end and distal portion to form a curved shape therebetween when the Nitinol basket is in an undeformed and deployed state, the proximal ends of the splines are configured to be grouped adjacent or near one another and to extend together in a proximal direction for incorporation into or onto a distal end or distal portion of a catheter body, and further wherein the splines are configured to be spaced approximately equal distances apart from one another when the Nitinol basket is in an undeformed and deployed state, and still further wherein the splines collectively form a basket shape when the Nitinol basket is in an undeformed and deployed state.

20. The method of claim 19, further comprising shaping and treating the splines such that, when the finished Nitinol basket is in an undeformed and deployed state, the basket shape is one of a spherical shape, a near-spherical shape, a tear-drop shape, a D-shape, a bulging asymmetric shape, and a shape where the splines are helically wound.

21. The methods of claim 19, further comprising drawing or forming the Nitinol tube or hypotube into a desired wall thickness comprising an initial outer diameter and a desired initial inner diameter.

22. The method of claim 19, wherein the cutting is performed by one or more lasers.

23. The method of claim 19, further comprising heat-setting the cut plurality of basket splines and distal tip while disposed in a first die or tool configured to hold or retain the basket splines and distal tip in first predetermined positions and orientations during the heat-setting step.

24. The method of claim 23, further comprising quenching the heat-set and cut plurality of basket splines and distal tip while still disposed in the die or tool configured to hold or retain the basket splines and distal tip in the first predetermined positions and orientations during the quenching step.

25. The method of claim 23, further comprising heat-setting and quenching at least once more the previously heat-set and cut plurality of basket splines and distal tip.

26. The method of claim 23, further comprising heat-setting and quenching at least once more the previously heat set and cut plurality of basket splines and distal tip while disposed in a second die or tool configured to hold or retain the basket splines and distal tip in second predetermined positions and orientations during the subsequent quenching step, the second die or tool being different from the first die or tool, and the second predetermined positions and orientations being different from the first predetermined positions and orientations.

27. The method of claim 19, wherein the single piece, slab or ingot comprising Nitinol has super-elastic properties associated therewith.

28. The method of claim 19, wherein super-elastic properties of the Nitinol basket are imparted thereto or enhanced therein by one or more of heat-setting steps and quenching steps.

29. The method of claim 23, wherein the Austenite finish temperature of the Nitinol basket is less than the interior temperature of a live human patient, thereby to preserve or enhance super-elastic properties associated with the Nitinol basket.

30. The method of claim 19, and after heat-setting and quenching steps, the Nitinol basket is subjected to one or more of media blasting, acid etching, and electropolishing.

31. The method of claim 19, further comprising imparting and forming an atraumatic shape, or providing an atraumatic covering or protective shield over, the distal tip, thereby preventing or minimizing injury to an interior surface of a patient's heart.

32. The Nitinol basket of claim 19, further comprising mounting or attaching a flex circuit on each spline, each flex circuit comprising a plurality of electrodes mounted on or attached thereto.

33. The Nitinol basket of claim 32, further comprising flowing, reflowing or reforming a polymeric material onto or into at least portions of each spline to at least one of hold, secure and register or orient each flex circuit and its electrodes thereon or thereto.
